(19) United States
(12) Patent Application Publication
(10) Pub. No.: US 2006/0074368 A1
(43) Pub. Date: Apr. 6, 2006

(54) Cover for port device

(76) Inventor: Sandra L. Palma, Fairfax, VA (US)

Correspondence Address: FLESHNER & KIM, LLP, P.O. BOX 221200, CHANTILLY, VA, 20153, US

(21) Appl. No.: 10/956,029

(22) Filed: Oct. 4, 2004

(51) Int. Cl. A61F 15/00

(52) U.S. Cl. 602/42

(57) ABSTRACT

A cover for a port inserted into a body of a patient is provided. The cover may include a substantially tubular band configured to completely surround a portion of a body of a patient where a port is inserted so as to substantially completely cover and secure the port in place when the port is not in use, and to provide ready access to the port without completely removing the band from the body portion when required.

### BACKGROUND OF THE INVENTION

1. Field of the Invention

This invention relates to a cover for a port device and associated line inserted into a patient.

2. Background of the Related Art

Ports are often inserted into the body of a patient to facilitate the administration of fluid and/or drug therapy, as well as for the drawing of fluids, such as blood, from a patient. These ports may be left in place so that medical personnel (or the patient) need only to access the port to administer fluids and/or draw samples, rather than relying on temporary methods, such as a needle and syringe, which may be uncomfortable and time consuming.

An example of one such body port is a peripheral intravenous central catheter (PICC). A PICC is a long, flexible tube that is inserted into a vein in a patient's arm and threaded up into a larger vein above the heart. Medications flowing into the PICC are mixed with and carried by the blood into the bloodstream. The PICC may be left in for extended periods of time for patients requiring repeated infusion type therapy. Although this arrangement facilitates the administration of fluid therapy when required, it also leaves the PICC exposed when not in use, leaving it vulnerable to damage and contamination.

Additionally, many patients may feel self conscious with an exposed PICC. Ace bandages are sometimes wrapped and secured around the port site, but these are bulky and uncomfortable to wear, do not provide ready access to the port when required, and are difficult to put on and secure with one hand. Stockinette-type webbing is also sometimes used, but its limited elasticity and web like structure prove uncomfortable and ill fitting, and do not prevent disturbance of the PICC.

### SUMMARY OF THE INVENTION

An object of the invention is to solve at least the above problems and/or disadvantages and to provide at least the advantages described hereinafter.

A cover for a port device according to embodiments of the invention is provided, including a substantially tubular band which surrounds a port insertion point on a body portion of a patient so as to cover and secure the port in place, and to provide ready access to the port as required.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 is a perspective view of a cover in accordance with an embodiment of the invention;

FIG. 2 is a partial perspective view of a cover in accordance with an embodiment of the invention showing an interior portion of the cover;

FIGS. 3A-3B are partial perspective views of a cover in accordance with an embodiment of the invention showing an interior portion of the cover;

FIG. 4 is a perspective view of a cover with an adjustment device in accordance with an embodiment of the invention; and

FIGS. 5A-5G illustrate a variety of wearing options for the covers shown in FIGS. 1-4 in accordance with embodiments of the invention.

### DETAILED DESCRIPTION OF PREFERRED EMBODIMENTS

The invention is directed to a cover for a port device. Certain embodiments of the invention are in the form of a substantially tubular cover configured to be inserted onto a body portion of a patient, and secured in place, while affording ready access to the port when required. The cover may be, for example, formed of an elastic material, which allows it to remain in place over the port. Further, the cover may be seamless, and may be formed of a knit or weave. The cover may also be formed of a material that is washable so that the cover may be washed and reused. Further, the cover may be secured in place over the port through a variety of mechanisms, including varying degrees of tightness in the cover, and securing mechanisms positioned at ends of the cover.

For example, the cover may include portions with a tighter knit or weave. Additionally, the cover may have transition portions between portions of different tightness of weave. Such tighter knit or weave portions allow for comfort to the wearer while guarding against slipping of the cover from its position over the port.

Alternatively, the cover may include elastic portions instead of or in addition to the tighter knit or weave portion. The cover may also include means for adjusting the tightness of the cover. In certain embodiments of the invention, this means can include a drawstring and pocket mechanism. The drawstrings may be, for example, fabric similar to the cover, another material, or an elastic material. The drawstring may include a tab securable to a securing portion with for example, a hook and loop type fastening mechanism, a hook and eye type fastening mechanism, a snap type fastening mechanism, or other fastening mechanism. Such mechanisms allow the tightness of the cover to be adjusted to suit a patient's particular needs and desired comfort level. Any combination of the above discussed configurations may be used to position and secure the cover over a port, thereby protecting the port and providing easy access to the port.

Further, the cover may be embellished with any number and type of designs to improve the outward appearance of the cover and enhance its use as an accessory when not covered by clothing. Additionally, these embellishments may include medical information related to the patient and/or the patient's medical information and treatment.

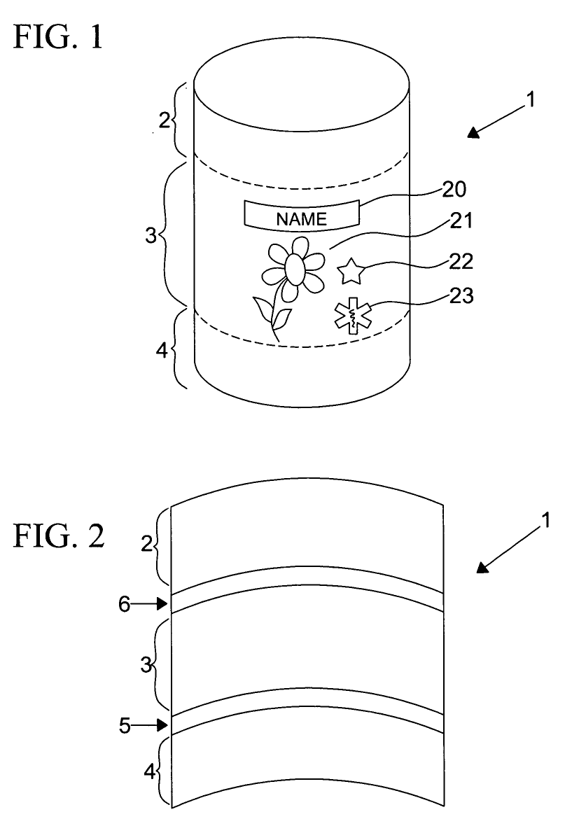

The invention will now be discussed referring to specific embodiments of the invention disclosed in FIGS. 1-5G of the present application.

FIG. 1 shows an embodiment of the invention wherein a cover for a port device, such as a PICC, is in the form of a band configured to be received onto an extremity of a patient, such as an arm or leg of a patient. Although a PICC may be referred to and used as an example, this is done merely for ease of discussion, and it is well understood that the invention as embodied and broadly described herein may be applied to any number and types of body ports and associated lines. The band 1 of FIG. 1 may have an elastic quality to allow the band to be slipped onto the extremity and positioned over the port. The band 1 may also be seamless. The band 1 of FIG. 1 is tubular in shape so as to easily fit onto and slide along an extremity of a patient. A lower portion 4 of the band 1 may be tighter fitting than an intermediate portion 3 or upper portion 2 of the band 1. Thus, when the band 1 is positioned covering a port, the more loosely fitting intermediate and upper portions 3 and 2 allow the band 1 to be easily slid and positioned over a port, while the tighter fitting lower portion 4 of the band 1 holds the band 1 securely in place. The band 1 may include embellishments, such as, for example, the designs 20-23 shown in FIG. 1 to enhance an outward appearance of the band 1 when it is not covered by clothing, or to convey medical information related to a patient's treatment. The band 1 may include embellishments, such as, for example, the designs 20-23 shown in FIG. 1 to enhance an outward appearance of the band 1 when it is not covered by clothing, or to convey medical information related to a patient's treatment.

In certain embodiments of the invention, the band 1 may be made of a relatively thin, snug fitting, seamless material so that clothing may be easily and comfortably worn over the band 1, if desired, and so that the band 1 does not interfere with other activities throughout the day. The band 1 may be embellished with any number and type of designs to improve the outward appearance of the band and enhance its use as an accessory when not covered by clothing. Additionally, these embellishments may include medical information related to the patient and/or the patient's medical information and treatment.

As set forth above, certain embodiments of the invention may have a tighter fit at a lower portion thereof. The tighter fit of the lower portion 4 may be achieved in a number of different ways. For example, a tighter knit or weave may be used in the area of the lower portion 4 of the band 1 than in either the intermediate or upper portions 3 and 2. Further, as shown in FIG. 2, there may be a first transition portion 5, which transitions the knit or weave from a tighter weave in the lower portion 4 to a looser weave in the intermediate portion 3, and a second transition portion 6, which transitions the knit or weave from the looser weave to a slightly tighter weave in the upper portion 2 so as to allow the upper portion 2 to remain in place once the band 1 is positioned over the port. The relatively tighter fit of the band 1 at the upper and lower portions 2 and 4 allows the band 1 to be easily slid onto an extremity such as, for example, an arm, and to comfortably and securely position the band 1 over a port, with just one hand, for example.

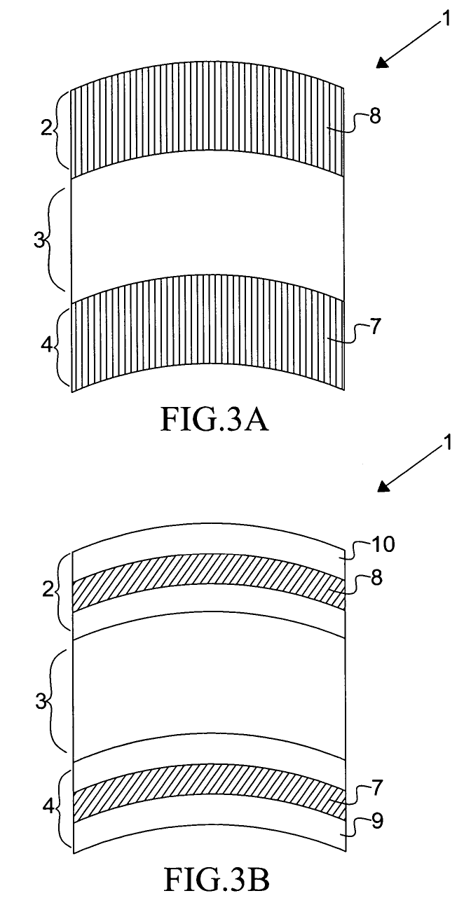

Referring to FIGS. 3A and 3B, the tighter fit of the band 1 at the upper and lower portions 2 and 4 may, alternatively, be achieved by providing elastic portions 7 and 8 at the upper and lower portions 2 and 4. The elastic portions 7 and 8 may be provided directly on inner surfaces of the upper and lower portions 2 and 4, as shown in FIG. 3A, in which case the elastic portions 7 and 8 would be in direct contact with a skin surface of a patient, thus providing additional guard against slipping. Alternatively, the elastic portions 7 and 8 may be formed on outer surfaces, or as shown in FIGS. 3A and 3B, the elastic portions 7 and 8 may be provided within pockets 9 and 10 formed in the upper and lower portions 2 and 4, potentially enhancing a comfort level in that area.

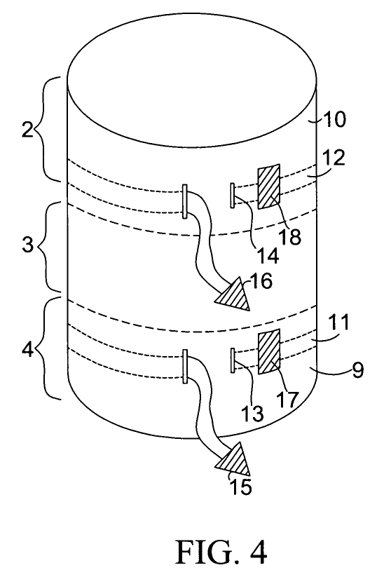

A patient may prefer to adjust a tightness of the upper and lower portions 2 and 4 of the band 1 themselves once the band 1 is positioned over the port. In this instance, adjustment devices may be provided for the upper and lower portions 2 and 4. For example, as shown in FIG. 4, the adjustment devices may include pockets 9 and 10 formed in the upper and lower portions 2 and 4 of the band 1, with drawstrings 11 and 12 running through the pockets 9 and 10. The drawstrings 11 and 12 may be fixed to the band 1 at ends 13 and 14, respectively, while tabs 15 and 16 provided at the other ends of the drawstrings 11 and 12 extend out from the pockets 9 and 10. The drawstrings 11 and 12 may be, for example, a fabric similar to that of the band 1. Alternatively, the drawstrings may be elastic, or may be any other material which is capable of sliding through the pockets 9 and 10. Tabs 15 and 16 at the ends of the drawstrings 11 and 12 may be affixed to securing portions 17 and 18 provided on inside or outside surfaces of the upper and lower portions 2 and 4 of the band 1 to adjust and secure a tightness of the band 1 once in place. The tabs 15 and 16 and securing portions 17 and 18 may be, for example, Velcro, hooks and eyes, snaps, or any other such fastening mechanism which can be manipulated, for example, with one hand and are particularly advantageous. Such mechanisms allow the tightness of the band 1 to be adjusted to suit a patient's particular needs and desired comfort level.

Any number of combinations for the configuration of the upper and lower portions 2 and 4 of the band 1 may be used to tighten and secure a position of the band 1 atop a port. For example, a tighter knit or weave in the upper portion 2 of the band 1 may be combined with elastic 7 or a drawstring 11 in the lower portion 4. Likewise, a tighter knit or weave in the lower portion 4 of the band 1 may be combined with elastic 8 or a drawstring 12 in the upper portion 2. These, and numerous other combinations, may all provide a patient with a comfortable cover band for a port which provides easy access to the port when required.

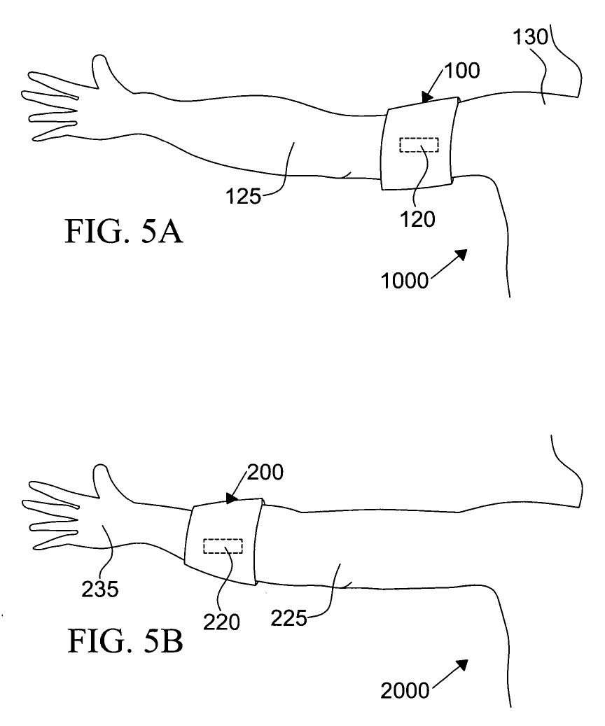

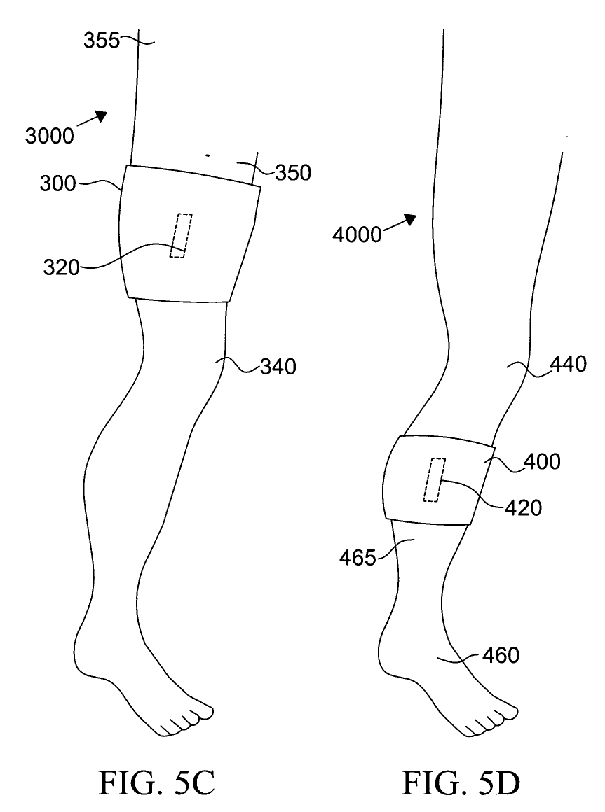

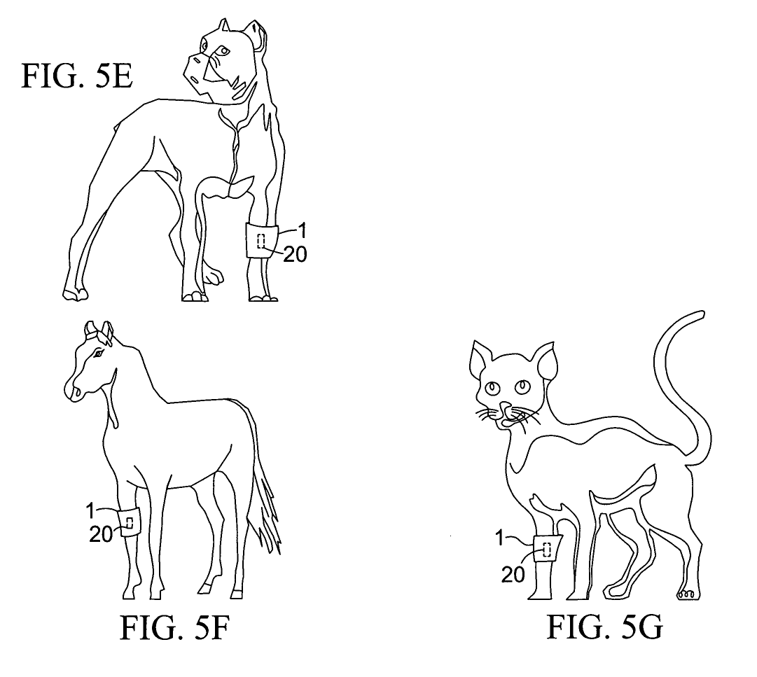

FIG. 5A shows an example in which a band 1 according to an embodiment of the invention is worn on an upper arm of a patient. In this example, the band 100 covers a port 120 which is inserted in an upper arm of a patient 1000, between the elbow 125 and the shoulder 130. FIG. 5B shows a band according to an embodiment of the invention worn on a lower arm of patient. The band 200 covers a port 220 inserted in a lower arm of a patient 2000, between the elbow 225 and the hand 235. FIG. 5C shows an example in which a band according to an embodiment of the invention is worn on an upper leg of a patient 3000. The band 300 covers a port 320 in the upper leg 350, or thigh, of a patient 3000, between the knee 340 and hip 355. FIG. 5D shows an example in which a band according to an embodiment of the invention is worn on a lower leg of a patient 4000. A band 400 covers a port 420 in the lower leg 465, or calf, of a patient 4000, between the knee 440 and the ankle 460. Thus, it is well understood that a cover according to embodiments of the invention may be positioned covering any location where a port is inserted, including but not limited to the lower arm, hand, upper leg/thigh, lower leg/calf, foot, etc. Likewise, in the examples shown in FIGS. 5A-5D, the band is worn by a human. However, the band may also be adapted to fit an animal, such as a dog, a cat, a horse, or any such animal with a port inserted into its extremity, as this type of infusion therapy is also common in animals. Examples of this are shown in FIGS. 5E-5G.

The foregoing embodiments and advantages are merely exemplary and are not to be construed as limiting the invention. The present teaching can be readily applied to other types of apparatuses. The description of the invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A cover for a port inserted into a body of a patient, comprising a substantially tubular band configured to completely surround a portion of a body of a patient where a port is inserted so as to substantially completely cover and secure the port in place when the port is not in use, and to provide ready access to the port without completely removing the band from the body portion when required.

2. The cover of claim 1, wherein the band comprises an upper portion provided along an upper edge portion of the band, a lower portion provided along a lower edge portion of the band, and an intermediate portion positioned between the upper portion and the lower portion.

3. The cover of claim 2, wherein the lower portion is configured to fit more tightly around a body portion of a patient than the intermediate portion.

4. The cover of claim 3, wherein the upper portion is configured to fit more tightly around a body portion of a patient than the intermediate portion.

5. The cover of claim 2, wherein the band comprises a knit or weave and the knit or weave of the band is tighter in the lower portion of the band than in the intermediate portion of the band, so as to provide a tighter fit in the lower portion of the band.

6. The cover of claim 5, wherein the knit or weave of the band is tighter in the upper portion of the band than in the intermediate portion of the band, so as to provide a tighter fit in the upper portion of the band.

7. The cover of claim 1, wherein the band has an elastic quality.

8. The cover of claim 2, wherein at least one of the upper or lower portions of the band comprises an elastic portion.

9. The cover of claim 8, wherein a first side of the elastic portion is affixed to an inner surface of the respective portion of the band, and a second side of the elastic portion is configured to contact a body portion of a patient when the band is worn by the patient.

10. The cover of claim 8, wherein the elastic portion is configured to be inserted into a pocket formed in the respective portion of the band.

11. The cover of claim 2, wherein a tightness of at least one of the upper and lower portions of the band is configured to be adjusted independent of a tightness of the other of the upper and lower portions of the band.

12. The cover of claim 11, wherein at least one of the upper and lower portions of the band comprises an adjustment device configured to adjust a tightness of the respective portion of the band independent of the other respective portion of the band.

13. The cover of claim 12, wherein the adjustment device comprises a pocket and drawstring combination adjustment device.

14. The cover of claim 13, wherein the pocket and drawstring combination adjustment device comprises:
a pocket formed in the respective portion of the band;
a drawstring fixed to the band at a first end and configured to be run through the pocket to an opening formed in the pocket, wherein a tab provided at a second end of the drawstring is configured to exit the pocket through the opening and to be secured to the band with a securing mechanism.

15. The cover of claim 14, wherein the securing mechanism comprises at least one of a hook and loop type fastening device, a hook and eye type fastening device, or a snap type fastening device configured to secure the tab to a securing portion fixed to the band.

16. The cover of claim 14, wherein the securing mechanism comprises at least one of a hook and eye type fastening device, a hook and eye type fastening device, or a snap type fastening device configured to secure the tab to the band.

17. The cover of claim 12, wherein the adjustment device is configured to allow a user to adjust a tightness of the respective portion of the band.

18. The cover of claim 2, wherein the lower portion of the band is configured to be moved away from the port so as to provide access to the port without removing the band.

19. The cover of claim 1, wherein the band comprises a knit or weave material.

20. The cover of claim 19, wherein the knit or weave of the material is continuous so as to form a seamless, tubular member.

21. The cover of claim 1, wherein the band further comprises embellishments on an outer surface thereof

22. The cover of claim 21, wherein the embellishments are decorative.

23. The cover of claim 21, wherein the embellishments comprise medical information.

24. The cover of claim 1, wherein the band is configured to be washed and reused.

25. A cover for a port inserted into a body of a patient, comprising a substantially tubular band configured to completely surround a portion of the body of the patient where the port is inserted, wherein the band comprises a lower portion configured to fit more tightly around the body portion than an intermediate portion of the band.

26. The cover of claim 25, wherein an upper portion of the band is configured to fit more tightly around the body portion than the intermediate portion of the band.

27. The cover of claim 26, wherein the band comprises a knit or weave and the knit or weave of the band is tighter in the lower portion of the band than in the intermediate portion of the band, so as to provide a tighter fit in the lower portion of the band.

28. The cover of claim 27, wherein the knit or weave of the band is tighter in the upper portion of the band than in the intermediate portion of the band, so as to provide a tighter fit in the upper portion of the band.

29. The cover of claim 25, wherein at least one of the upper or lower portions of the band comprises an elastic portion.

30. The cover of claim 29, wherein a first side of the elastic portion is affixed to an inner surface of the respective portion of the band, and a second side of the elastic portion is configured to contact a body portion of a patient when the band is worn by the patient.

31. The cover of claim 29, wherein the elastic portion is configured to be inserted into a pocket formed in the respective portion of the band.

32. The cover of claim 26, wherein a tightness of at least one of the upper or lower portions of the band is configured to be adjusted independent of a tightness of the other of the upper and lower portions of the band.

33. The cover of claim 26, wherein at least one of the upper and lower portions of the band comprises an adjustment device configured to adjust a tightness of the respective portion of the band independent of the other respective portion of the band.

34. The cover of claim 33, wherein the adjustment device comprises a pocket and drawstring combination adjustment device.

35. The cover of claim 33, wherein the pocket and drawstring combination adjustment device comprises:
a pocket formed in the respective portion of the band;
a drawstring fixed to the band at a first end and configured to be run through the pocket to an opening formed in the pocket, wherein a tab provided at a second end of the drawstring is configured to exit the pocket through the opening and to be secured to the band with a securing mechanism.

36. The cover of claim 35, wherein the securing mechanism comprises at least one of a hook and loop type fastening device, a hook and eye type fastening device, or a snap type fastening device.
